(19) United States
(12) Patent Application Publication
(10) Pub. No.: US 2018/0092644 A1
(43) Pub. Date: Apr. 5, 2018

(54) ELASTIC BAND LIGATION DEVICE WITH INSERTION CAP AND RELIEF VALVE

(71) Applicant: Clinton G. Wallis, Nichols Hills, OK (US)

(72) Inventor: Clinton G. Wallis, Nichols Hills, OK (US)

(21) Appl. No.: 15/726,208

(22) Filed: Oct. 5, 2017

Related U.S. Application Data
(60) Provisional application No. 62/404,244, filed on Oct. 5, 2016.

(51) Int. Cl. A61B 17/12

(52) U.S. Cl. CPC A61B 17/12; A61B 2017/306

(57) ABSTRACT

An elastic band ligation device for treating hemorrhoids with an invertible insertion cap is provided herein. The device includes an inner tubular member, and outer tubular member, and a plunger configured with an insertion cap that covers the proximal end of the device during the insertion portion of a hemorrhoid ligation procedure. The insertion cap is fully withdrawn into the inner tubular member during the hemorrhoid tissue isolation portion of the procedure. Movement of an outer tubular member relative to the inner tubular member will push the elastic band from the inner tubular member.

### CROSS REFERENCE TO RELATED APPLICATION

This application claims priority from U.S. Provisional Application 62/404,244 filed Oct. 5, 2016.

The present disclosure relates to a device used in the medical treatment of hemorrhoids using a procedure known as elastic band ligation. More specifically, the disclosure relates to an elastic band ligation device that uses suction to aide in the banding process.

### BACKGROUND

Elastic band ligation is a commonly-used, nonsurgical procedure performed in the field of treating hemorrhoids. The procedure involves isolating a hemorrhoid within the patient's rectum and placing an elastic band around the “neck” (isthmus) of the hemorrhoid tissue. This banding will constrict blood flow to the tissue, causing it to degenerate, and result in both the band and the hemorrhoid eventually sloughing off and passing out of the rectum during a bowel movement. At the point of banding, the surrounding tissue hardens and future hemorrhoid tissue prolapse is minimized, thereby not only avoiding a surgical procedure but also preventing the reoccurrence of hemorrhoids at the location where banding occurred.

Multiple devices exist that utilize elastic band ligation and many of these devices accomplish the procedure using suction as the method to isolate the hemorrhoid tissue desired for banding. Examples include U.S. Pat. No. 5,741,273 to O'Regan; U.S. Pat. No. 5,203,863 to Bidoia; and U.S. Pat. No. 8,303,605 B2 to Bastia. Such ligation devices attempt to ease patient discomfort through the inclusion of obturators and anti-pinch wipers, intending to prevent tissue from becoming drawn between the inner tubular member and the obturator. U.S. Pat. No. 9,101,360 B2 discloses one such device. Moreover, in an effort to reduce patient discomfort, variations on the suction tip angle, U.S. Pat. No. 4,257,419 to Goltner et al., and device curvature, U.S. Patent Application 2014/0121679 A1 to Cleator, and the like have been explored.

None of these devices or improvements thereupon, however, solves two very specific sources of patient discomfort created by using the devices themselves, “scraping” and “pinching.” The “scraping” sensation occurs during device insertion, which is caused by the various diameter changes of the device and the presence of an exposed elastic band. The “pinching” sensation occurs during and post procedure and is attributable to banding too much tissue. Although the device may band an unnecessary amount of mucosa tissue without causing patient discomfort, it is also possible to inadvertently band muscularis tissue, which causes significant patient discomfort.

Inherent to previously developed band ligation devices, a pusher and an elastic band are exposed to the sensitive anus and rectum tissue prior to the instrument reaching the location of the hemorrhoid intended for banding. This exposure can cause significant “scraping” discomfort during the procedure. Additionally, many previously developed devices lack either a means for regulating or a means for indicating how much tissue is drawn into the device for banding. This results in a band and check process, whereby the physician must verify with the patient whether the procedure has banded too much tissue. If so, the patient will experience a “pinching” sensation and adjustments may be made by the physician, causing the patient further discomfort and prolonging the procedure.

### SUMMARY

The current disclosure is directed to an elastic band ligation device for the medical treatment of hemorrhoids, configured to ease patient discomfort during insertion and prevent the banding of excessive tissue during the procedure. The device comprises an inner tubular member, an outer tubular member, and a plunger. The plunger is movable in the inner tubular member, and is configured to create negative pressure within the inner tubular member, thereby drawing hemorrhoid tissue into the inner tubular member. The inner tubular member, the outer tubular member and the plunger all have a first, or proximal, and second, or distal, end. The inner tubular member proximal end is configured with an inner tubular member opening, allowing the proximal end of the plunger disposed within the inner tubular member to pass through said inner tubular member opening in both proximal and distal directions. The opening at the proximal end may be referred to as a first, or proximal opening. The inner tubular member proximal end is further configured to retain an elastic band, which will slide onto the outer surface of the inner tubular member using conventional means readily known to those skilled in the art. The inner surface of the inner tubular member proximal end is configured to accept hemorrhoid tissue passing through said inner tubular member opening. The inner surface of the inner tubular member at the distal end is configured to house and retain the plunger, and the outer surface of the inner tubular member distal end is configured with a handle, allowing the physician to securely hold the inner tubular member.

The inner tubular member is disposed in the outer tubular member, and the outer tubular member maybe configured to come in contact yet easily slide along the outer surface of the inner tubular member. When sliding proximately (i.e. toward the opening at the proximal end of the inner tubular member) in one embodiment the outer tubular member comes into contact with the elastic band positioned on the proximal end of the inner tubular member or the elastic band past the proximal end of the inner tubular member. The direction from the distal to the proximal end may be referred to as the forward direction. Thus, forward movement of the outer tubular member will, in one embodiment, push the elastic band off of the inner tubular member to band the tissue. The distal end of the outer tubular member has a handle capable of accepting single-hand operation.

The plunger has an insertion cap at the first, or proximal end thereof. The insertion cap is configured to pass into the proximal end of the inner tubular member. The plunger has a means for creating a seal against the inner surface of the inner tubular member when the plunger is pulled through the inner tubular member. As non-limiting examples, the sealing means may comprise a solid feature with a sealing ring disposed thereupon or it may comprise a sealing member disposed on the plunger stem. Negative pressure is created when hemorrhoidal tissue creates a seal against the proximal opening of the inner tubular member. The distal end of the plunger is configured with a handle means extending distally from the distal end of the inner tubular member.

In one embodiment the insertion cap is composed of a silicon, latex, or similarly flexible material. In one embodiment, the insertion cap covers the inner tubular member proximal end, elastic band, and the outer tubular member proximal end. The insertion cap is further configured to be completely withdrawn into the inner tubular member upon pulling the handle of the plunger in a distal direction, thereby completely uncovering the outer tubular member, elastic band, and outer surface of the inner tubular member proximal end. The outer tubular member can then engage and deploy the elastic band. The negative pressure generated in the inner tubular member may be regulated by a relief valve which in one embodiment is defined on the inner tubular member wall. As a result a maximum pressure to be generated as a result of the plunger being withdrawn may prevent, or at least alleviate the possibility of banding too much hemorrhoid tissue.

In another embodiment, the insertion cap may be configured with a variety of hole and slit configurations to facilitate being withdrawn into the inner tubular member. Moreover, the insertion cap may be configured using multiple materials, a material of variable density, or varying material thickness configured to achieve the desired inversion properties. In one embodiment the insertion cap in an insertion position will cover the elastic band and the end of the outer tubular member to create a smooth insertion surface with no abrupt edges or abrupt diameter changes that might cause scraping upon insertion.

In various other embodiments, the relief valve may be located within the sealing means of the plunger, or may be located within a plunger that is configured with a hollow channel, allowing the relief valve to be disposed within the plunger and manually rendered ineffective by covering the relief valve itself or similarly preventing the valve's operation. The relief valve may also be located on a stem or air tube extending from the inner tubular member. Those skilled in the art will recognize the location of the valve is not strictly defined. The relief valve simply is to be connected to the negative pressure generated inside the inner tubular member regardless of the location of said valve.

In yet another embodiment, the relief valve may be removable in order to allow different relief valves to be selected based upon procedural conditions. Those skilled in the art will recognize that a variable valve will accomplish the same result as discrete interchangeable relief valves.

In yet another embodiment, either the inner tubular member or the plunger is configured to indicate, either via marking or a physical interference means, the location of the plunger at which the insertion cap is completely withdrawn beyond the proximal opening of the inner tubular member. One embodiment of the invention will be made from various plastic materials and considered disposable by those skilled in the art.

The elastic band ligation device prevents “scraping” by covering the various diameter steps between the inner tubular member, elastic band, and outer tubular member through the use of the insertion cap. The device also provides for a maximum amount of negative pressure to be generated within the inner tubular member, preventing the “pinching” caused by banding too much tissue.

In an additional embodiment, an outer tubular member has a first or proximal end spaced from a movable pusher ring that is disposed about an inner tubular member. The outer tubular member is tethered to the pusher ring. A pull applied to the outer tubular member will cause the tether to move the pusher ring in a proximal direction. The pusher ring will engage and push an elastic band off of the inner tubular member to band the tissue. In this way, the ligation device can be inserted and tissue banded, with one hand. Thus, the ligation device may be referred to as a single hand operated ligation device. The ability to perform ligation with one hand leaves the operator a free hand to perform any other necessary tasks. The insertion cap will cover the elastic band, proximal at the inner tubular member, and at least the forward, or proximal edge of the pusher ring.

### BRIEF DESCRIPTION OF THE DRAWINGS

FIG. 1 is a perspective view of a first embodiment as assembled and ready for insertion into the patient.

FIG. 2 is a section view indicating the components of the invention of FIG. 1.

FIG. 3 is a broken section view of the proximal end of the first embodiment of the invention.

FIG. 4 is a detailed section view of a first embodiment relief valve location.

FIG. 5 is a section view depicting a first embodiment of the present invention with the plunger in the withdrawn position.

FIG. 6 is a broken cutaway of the proximal end of a first embodiment drawing hemorrhoid tissue into the inner tubular member.

FIG. 7 is a perspective view of a first embodiment with the plunger in the withdrawn position as it would after the hemorrhoid tissue drawing phase of the procedure.

FIG. 8 is a perspective view of a second embodiment of the invention.

FIG. 9 is a cross sectional view of the second embodiment.

FIG. 10 is a close up view in cross section of the proximal end of the second embodiment.

FIG. 11 is a close up view in cross section of a central portion of the second embodiment with the plunger withdrawn.

FIG. 12 is a cross section view of the second embodiment with the plunger withdrawn and the insertion cap drawn into the inner tubular member. The tether is not shown in connection to the pusher ring in FIG. 12.

FIG. 13 is a close up view in cross section of the proximal end of the second embodiment with the insertion cap withdrawn.

FIG. 14 is a perspective view of the second embodiment with the plunger withdrawn.

FIG. 15 is a cross section showing the insertion cap withdrawn, and the band pushed from the inner tubular member.

FIG. 16 shows the outer tubular member in a locked position.

FIG. 17 shows the outer tubular member rotated to the unlock positon, and pulled to cause the pusher ring to push the elastic band from the inner tubular member.

FIGS. 18 and 19 are views of an alternative embodiment of a plunger with a valve opening therein.

FIGS. 20 and 21 are views of an additional embodiment and a stem with an alternative location for a valve opening.

FIG. 22 is an alternative embodiment of an insertion cap with a hole and slot configuration.

FIG. 23 is a close up section view of the connection of the connection of a tether to a pusher sleeve.

### DETAILED DESCRIPTION

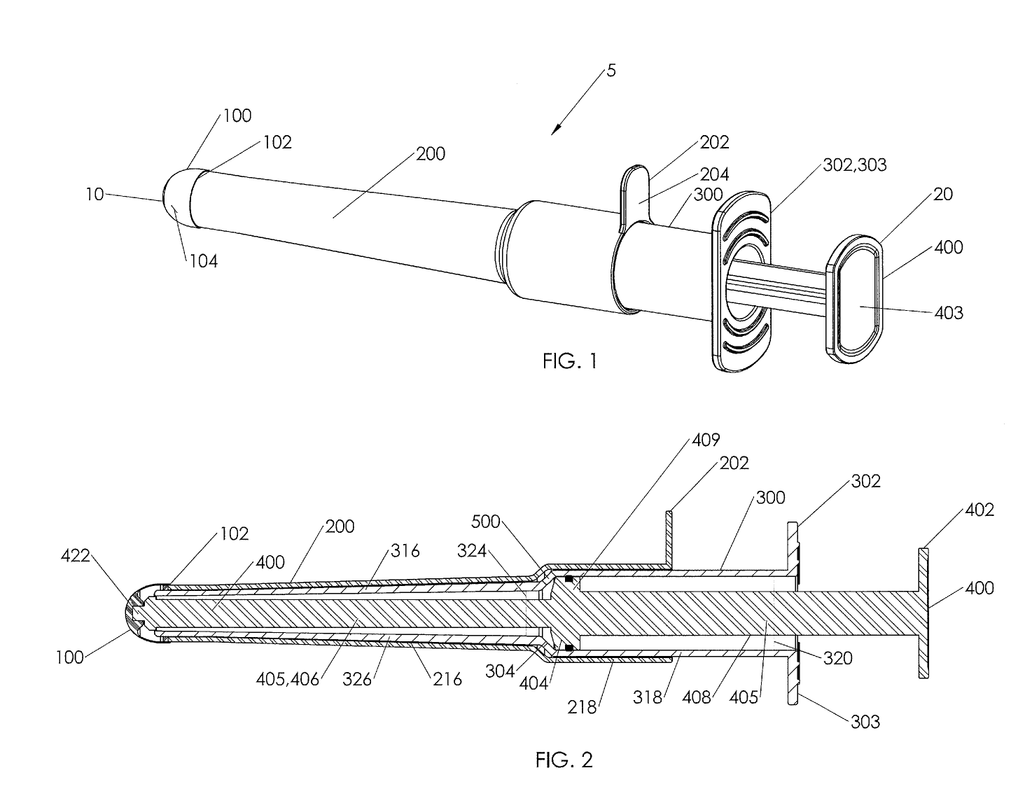

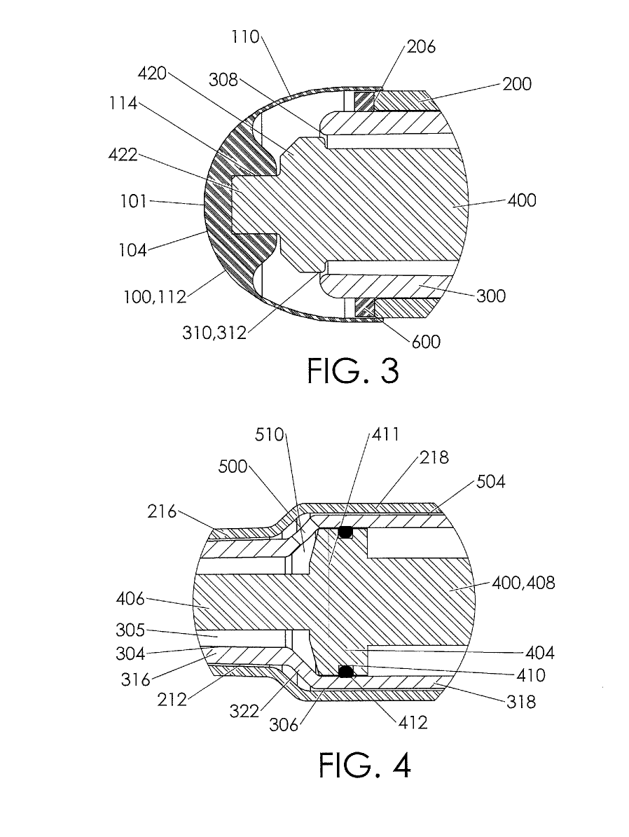

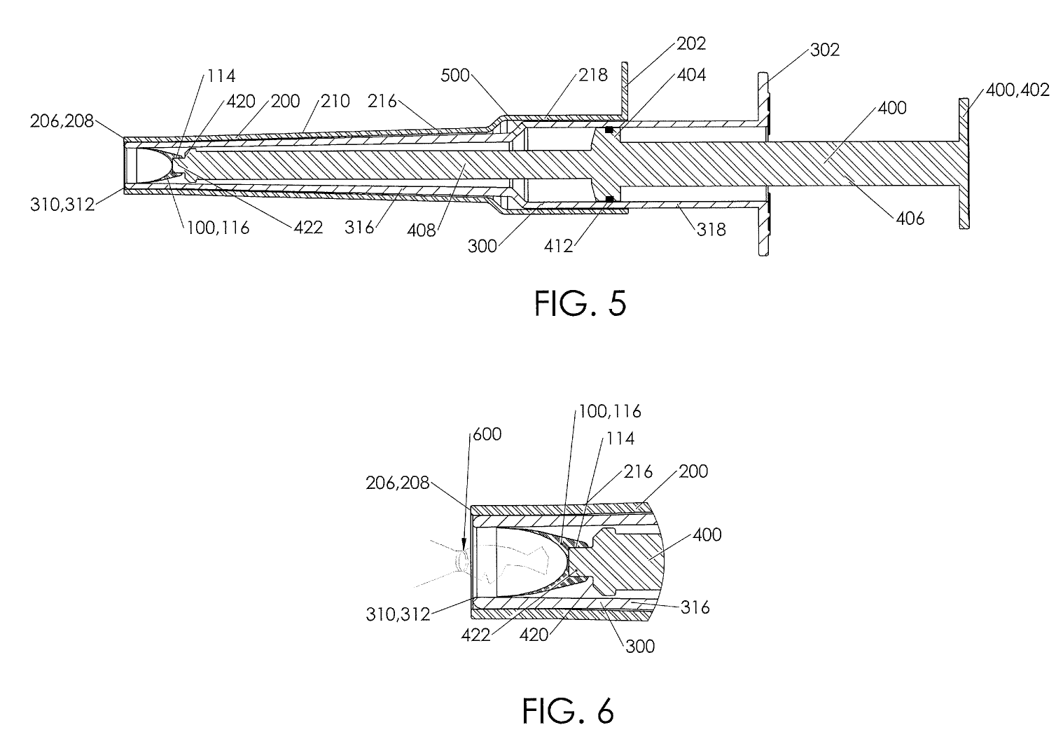

Referring to the drawings, FIG. 1 is a perspective view of a first embodiment of a ligation device 5 as assembled and prepared for performing elastic band ligation. Ligation device 5 has a first, or proximal end 10 and a second or distal end 20. Ligation device 5 comprises an inner tubular member 300, an outer tubular member 200, and a plunger 400 that is configured with an insertion cap 100. Insertion cap 100 has an insertion or proximal tip 101 and an edge 102 at the distal end thereof. An exterior surface 104 of the insertion cap is curved so that any discomfort to the patient can be minimized upon insertion. An inner surface 110 of insertion cap has a curvature such that upon inversion from the insertion shape 112, which may be referred to as an insertion position as shown in FIGS. 1 and 2 to the inverted or retracted shape, or position 116 as shown in FIG. 5 and FIG. 6, there will be no damage to insertion cap 100. The insertion cap 100 has a receptacle 114 which will receive a stem on plunger 400 as will be explained in more detail herein. When insertion cap 100 is in the insertion position 112 it covers the edges of the inner tubular member 300 and outer tubular member 200 at the proximal ends thereof to create a smooth insertion surface.

The outer tubular member 200 has handle or handle means 202 at the distal or second end 204 thereof. Outer tubular member 200 defines an opening 206 at the proximal or first end 208 thereof. Inner tubular member 300 extends through opening 206 at the proximal end 208 of outer tubular member 200. Outer tubular member 200 has outer surface 210 and inner surface 212. Outer tubular member comprises a first tapered section 216 and a second section 218. Second section 218 is generally cylindrical, while first section 216 tapered and becomes smaller in diameter toward proximal end 208. Inner tubular member 300 is closely received in outer tubular member 200 and more specifically is closely received by inner surface 212 of outer tubular member 200.

Inner tubular member 300 has handle or handle means 302 at the second or distal end 303 thereof. Inner tubular member 300 has inner surface 304 and outer surface 306 which is received in outer tubular member 200. Inner surface 304 defines an interior 305 of inner tubular member 300. A radius 308 at the first or proximal end 310 of inner tubular member 300 is defined on the inner surface 304 thereof. The radius 308 will be shaped or configured to prevent any catching or damage of the insertion cap 100 as it moves from the insertion position 112 to the retracted or inverted position 116. An opening 312 is defined at the first or proximal end 310 and may be referred to as first or proximal opening 312. Inner tubular member 300 comprises a first tapered section 316 and a second section 318. Second section 318 is generally cylindrical, while first section 316 tapers and becomes smaller in diameter toward proximal end 310. Inner tubular member 300 has a diameter 320 in second section 318 and has a transition section 322 between first section 316 and second section 318. The distal end of first section 316 has an inner diameter 324 which is smaller than inner diameter 320. The inner surface 304 tapers inwardly in first section 316 from transition section 322 to proximal opening 312. Inner surface 304 thus defines an angled wall 326 in first section 316.

Plunger 400 has a handle, or handle means 402 at the distal or second end 403 thereof. A seal 404, which may be referred to as a sealing ring 404, is defined on a plunger, or plunger stem 405. Stem 405 has first or proximal portion 406 and second or distal portion 408. Sealing ring 404 may comprise a generally circular body 409 with an outer diameter 411. The outer diameter 411 has a groove 410 defined therein which receives a sealing ring, for example, an O ring 412. O ring 412 will seal against the inner surface 304 in the second section 318 of inner tubular member 300. When handle 402 is pulled to withdraw the insertion cap 100 into inner tubular member 300 seal 404 will create negative pressure so that tissue will be drawn into the inner tubular member 300. In alternate embodiments the sealing means 404 may consist of a plunger or rubber section disposed around the location where the proximal 406 and distal 408 plunger stems meet.

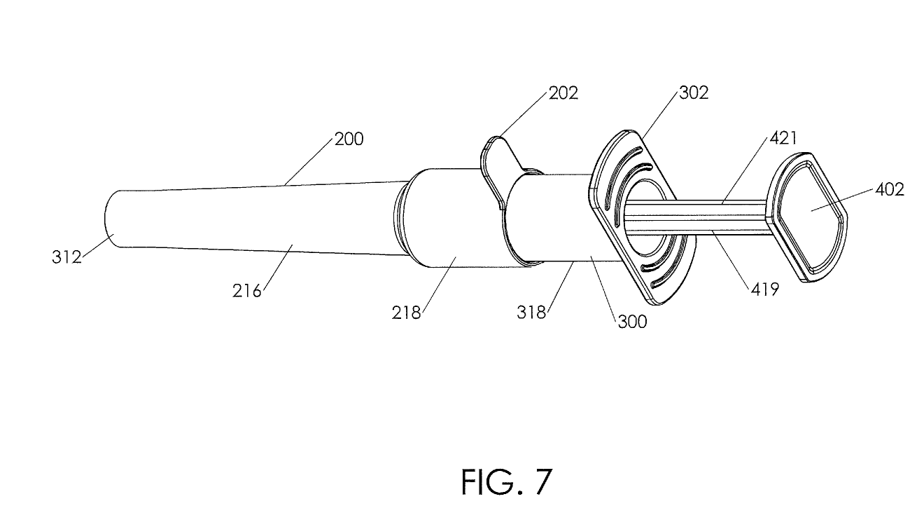

The stem 405 may be configured as depicted in FIG. 7 in a generally T shaped configuration with legs 419 and 421 that are generally perpendicular to one another. Stem 405 may comprise other shapes that provide a space inside inner tubular member 302. A head 420 is positioned at proximal end 416. Head 420 has a connecting stem 422 extending therefrom to be inserted into receptacle 114 on insertion cap 100.

Inner tubular member 300 has a relief valve 500 in transition section 322. A vent gap 504 allows air to pass therethrough when the plunger 400 is withdrawn. Relief valve 500, which may be referred to as a valve opening 500, is communicated with an exterior of the ligation device 5 through vent gap 504. The depicted embodiment creates an air chamber 510 bounded by the inner tubular member inner surface 304, the sealing means 404 and proximal opening 312. An elastic band 600 is disposed about inner tubular member 300 near proximal end 310 thereof. Elastic band 600 will be engaged by outer tubular member 200 and will be pushed beyond proximal end 310 of inner tubular end 300 to band hemorrhoidal tissue.

Air is drawn into the inner tubular member 300 through valve opening 500 when plunger 400 is withdrawn. Because air is pulled through valve opening 500, suction through proximal end 310 is restricted to an amount less than that which occurs in a device without such a valve opening. In this way the amount of suction can be limited to at least alleviate the problem of banding too much tissue with elastic band 600.

As depicted in the cross-section of FIG. 2, insertion cap 100 is connected to plunger proximal stem 406. In one embodiment, insertion cap 100 is made of a flexible material, like silicon, latex, or any similarly flexibly suitable material. The exterior curvature of the insertion cap 100 is configured to maximize patient comfort, generally a radius shape that will not inhibit the inner surface 110 from freely inverting and withdrawing into proximal opening 312. The insertion cap 100 is configured to conform to the underlying structures of the plunger 400, the inner tubular member 300, the elastic band 600, and the outer tubular member 200, creating a relatively smooth contour over the underlying structures during the ligation procedure. As a result, insertion cap 100 covers the proximal end 310 of inner tubular member 300, the proximal end 208 of outer tubular member 200 and elastic band 600. Distal edge 102 thus extends beyond the inner tubular member proximal opening 312, beyond the elastic band 600 and beyond the proximal end 208 of the outer tubular member 200. The material of the insertion cap 100 is selected and the inner surface 110 is contoured such that the insertion cap 100 freely slides along each aforementioned underlying structure. Inner surface 110 of the insertion cap 100 is configured such that when the insertion cap 100 is withdrawn distally past proximal opening 312 along the inner surface of the inner tubular member 304, the insertion cap inner radius does not separate, fracture, or experience material stresses sufficient to damage the insertion cap 100. Insertion cap 100 is thus an invertible insertion cap the will invert when plunger 400 is withdrawn.

Prior art ligation devices include a solid obturator as opposed to the invertible cap described in the current disclosure. The solid obturator in prior art devices was positioned inside the inner tubular member, and thus allowed for the edges of both the inner tubular member and the outer tubular member to scrape the tissue of a patient upon insertion into the rectum of a patient. Upon withdrawal of the prior art obturator into the inner tubular member, tissue can be trapped between the inner tubular member and the obturator to pinch tissue, causing unnecessary discomfort to the patient. The ligation device described herein alleviates both of those issues. Invertible cap 100 covers the edges of the inner and outer tubular members 300 and 200 in the insertion position to prevent scraping, and when withdrawn will invert and be pulled into the inner tubular member 300, to prevent the pinching of tissue.

FIG. 2 depicts ligation device 10 with plunger 400 in its most proximal position. In this position, the ligation device 10 is in the insertion position ready for insertion into a patient. During operation, and as depicted in FIG. 5, insertion cap 100 will invert to inverted, or retracted position 116 when withdrawn past proximal opening 312 into the inner tubular member 300. Hemorrhoid tissue will be drawn into the inner tubular member 300 through opening 312 as the plunger 400 is withdrawn as a result of seal 404 engaging inner tubular member 300. Valve opening 500 will limit the suction to prevent unwanted extra tissue from being pulled in to the inner tubular member 300. FIG. 6 depicts ligation device 10 whereby hemorrhoid tissue has been drawn into the inner tubular member 300 through the proximal opening 312. The inverted insertion cap 100 is configured so that no seal is created against the inner surface 304 of the inner tubular member when pulled by the proximal stem 406 of plunger 400. Elastic band 600 is pushed past the proximal opening 312 with the outer tubular member 200 to band the hemorrhoid tissue. FIG. 6 shows the position of outer tubular member 200 after it has been moved to push the band 600 from inner tubular member 300.

An alternate embodiment of an insertion cap is depicted in FIG. 21. Alternate embodiment 100a includes a plurality of holes 118 disposed thereabout. Holes 118 are connected to slots 120 such that the transition is a gradual curve 122. Additionally, the inner surface of the insertion cap 110a is concave between adjacent holes 118, to facilitate the cap inversion when being drawn into the inner tubular member 300.

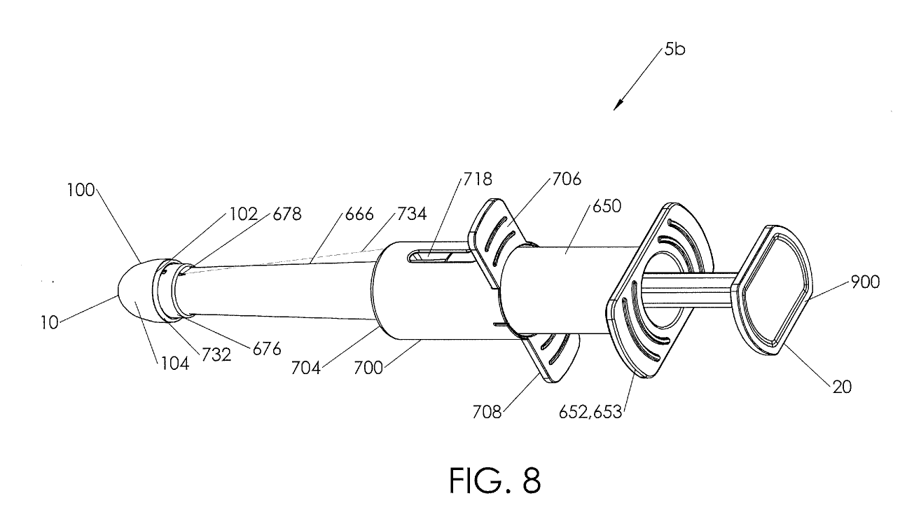

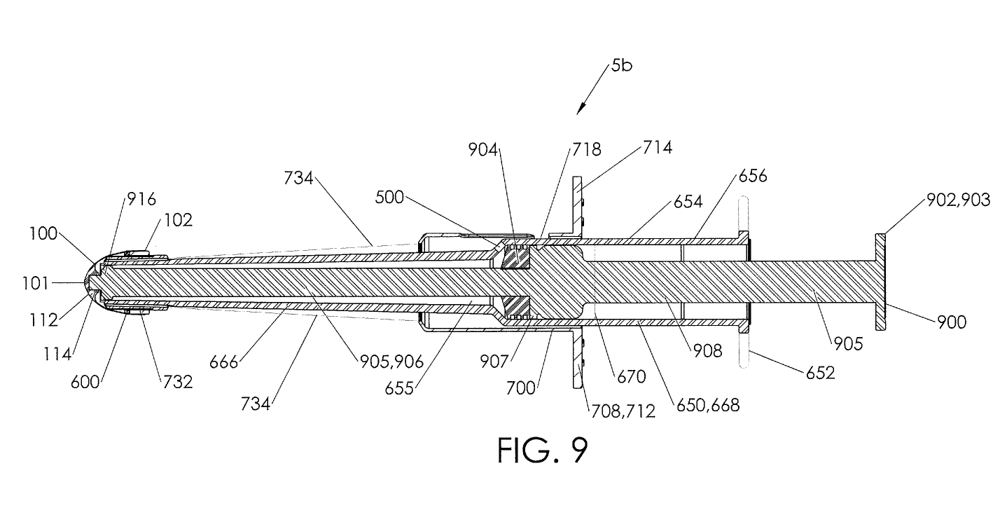

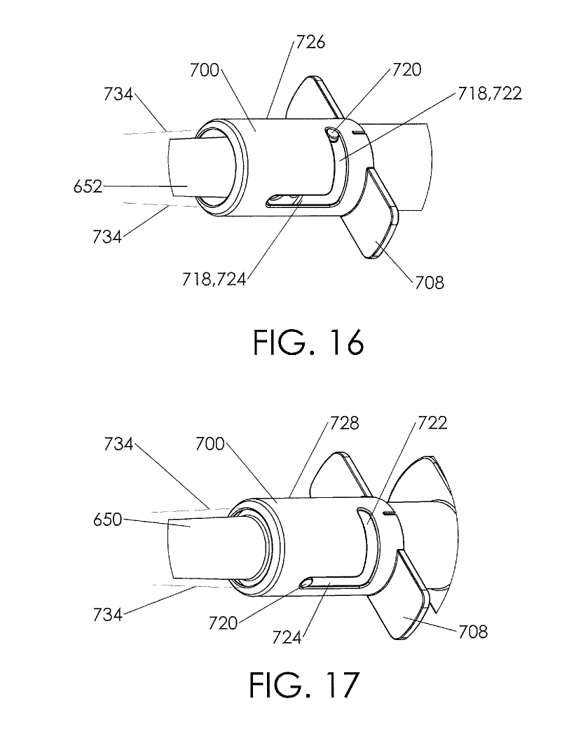

An additional embodiment of a ligation device 5b is shown and depicted in FIGS. 8-16. The ligation device of FIG. 9 is similar to ligation device 5. However, with ligation device 5b the outer tubular member 700 is pulled distally relative to inner tubular member 650 to push elastic band 600 from the inner tubular member 650 to band hemorrhoidal tissue. Outer tubular member 700 has a first, or proximal end 704 that is spaced from the location of the elastic band 600.

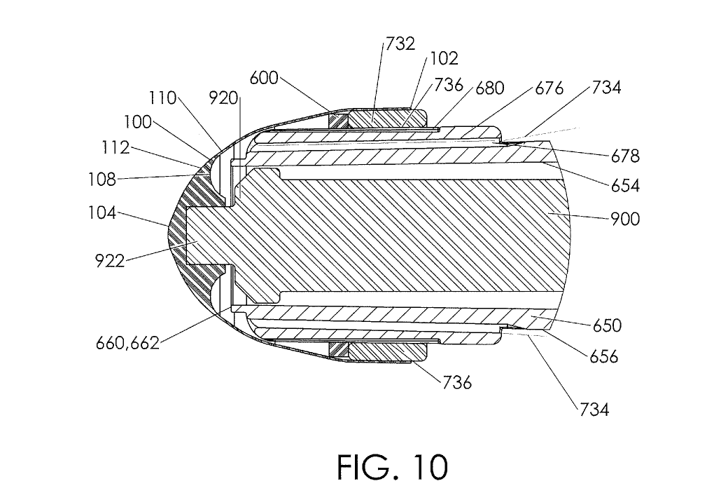

In the embodiment of FIG. 8 inner tubular member 650 is similar to inner tubular member 300. Inner tubular member comprises a handle 652 at the second or distal end 653 thereof. Inner tubular member 650 has inner surface 654 and outer surface 656 which is received in an outer tubular member 700. Inner surface 654 defines an interior 655 of inner tubular member 650. Inner tubular member 650 has a proximal end 660 that defines a proximal opening 662. Proximal end 660 is shown in detail in FIGS. 10 and 13 in the insertion and retraction positions respectively. In the retracted position, hemorrhoidal tissue will be pulled in the proximal opening 662.

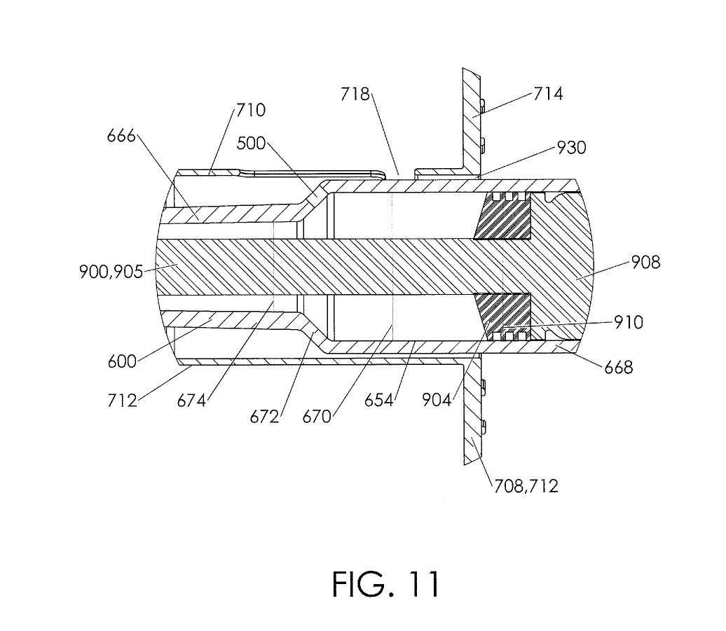

Inner tubular member has a first tapered section 666 and second section 668. Second section 668 is generally cylindrical, while first section 666 tapers and becomes smaller in diameter towards proximal end 660. Inner tubular member 650 has a diameter 670 in second section 668 and has a transition section 672 between first section 666 and second section 668. Valve opening 500 may be defined in transition section 672. The distal end of first section 666 has an inner diameter 674 which is smaller than inner diameter 670. Outer surface 656 may be stepped radially outwardly to a shoulder 676 at or near the proximal end thereof. A slot 678 is defined at the proximal end of inner tubular member 650 and passes through shoulder 676. The slot 678 is designed to receive a tether as will be explained in more detail herein below. A groove 680 is defined in the outer surface 656 of inner tubular member 650 on shoulder 676 and will allow the tether to pass therein and ultimately be attached to a movable pusher ring as shown in FIG. 11.

Outer tubular member has a distal end 706 with a handle 708. Handle 708 has two handle portions 712 and 714 so that it can be manipulated easily. Outer tubular member 700 has inner surface 710 and outer surface 716. An L-shaped locking slot 718 is defined in outer tubular member 700. An anchor 720 is attached to and extends from inner tubular member 650 and is received in L-shaped locking slot 718. Locking slot 718 has a circumferential leg or portion 722 and a longitudinal leg 724. Outer tubular member 700 of ligation device 5b is in a locked position 726 when anchor 720 is received in circular leg portion 722. The locked position is the insertion position of ligation device 5b. Outer tubular member 700 may be rotated such that anchor 720 is moved into longitudinal leg 724 which is the unlocked position 728 of the outer tubular member 700. In the unlocked position the outer tubular member 700 can be pulled distally relative to the inner tubular member 650.

A moveable pusher ring 732 is disposed about inner tubular member 650. A taper on inner tubular member 650 will prevent the pusher ring 730 from moving distally thereon past the desired location. A stop ring may be used to hold pusher ring 732 in place if desired. Moveable pusher ring 732 will be tethered to outer tubular member 650. In the embodiment shown at least one tether 734 will pass through slot 678 and groove 680 and will connect to a pusher ring 732, which may also be referred to as a pusher sleeve 732. In the embodiment described and shown, there are two tethers 734, both of which connect to an inner surface of pusher ring 732. Tethers 734 will pass through slot 678 and will wrap around proximal end 662. Tethers 734 will then pass through groove 680 so they may be connected at connection points 736 to movable pusher sleeve 732. Tethers 734 may be spaced circumferentially 180 degrees. When connected in the manner described the pulling action of outer tubular member 700 distally relative to inner tubular member 650 will simultaneously cause moveable pusher ring 732 to be pushed toward band 600, moving band 600 off of inner tubular number 650 and on to the tissue to be banded.

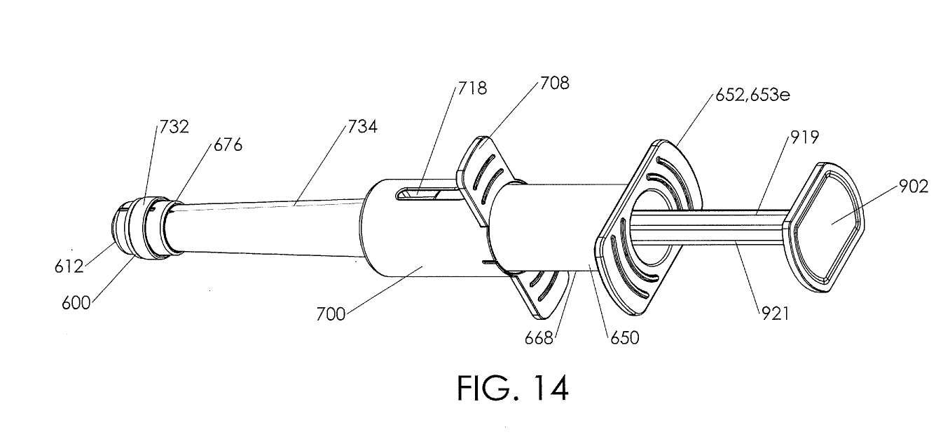

A Plunger 900 in the embodiment of FIG. 8 has a handle 902 at the distal end 903. A seal 904, which may be referred to as sealing means 904 is mounted on a plunger stem 905. Seal 904 may comprise a rubber sealing element that engages inner surface 654 of inner tubular member 650. Stem 905 has first or proximal portion 906 and second or distal portion 908. A seal platform 907 may be a part of stem 905 to mount seal 904 thereto. Outer diameter 910 of seal 904 will engage and seal against inner surface 654 of inner tubular member 650. Stem 905 may have the generally T-shaped configuration shown in FIGS. 8 and 14 with legs 919 and 921. Head 920 is positioned at the proximal end 916 of plunger 900. Valve opening 500 is positioned in transition section 672 of inner tubular member 650 and is therefore open to the exterior of atmosphere through a passage 930 defined by outer tubular member 900 and inner tubular member 650. Connecting stem 922 will be received in slot 114 on insertion cap 100. Air is drawn into inner tubular member 650 through valve opening 500 when plunger 900 is withdrawn. As a result, suction through proximal end 660 is restricted to an amount less than that which occurs in a device without a valve opening.

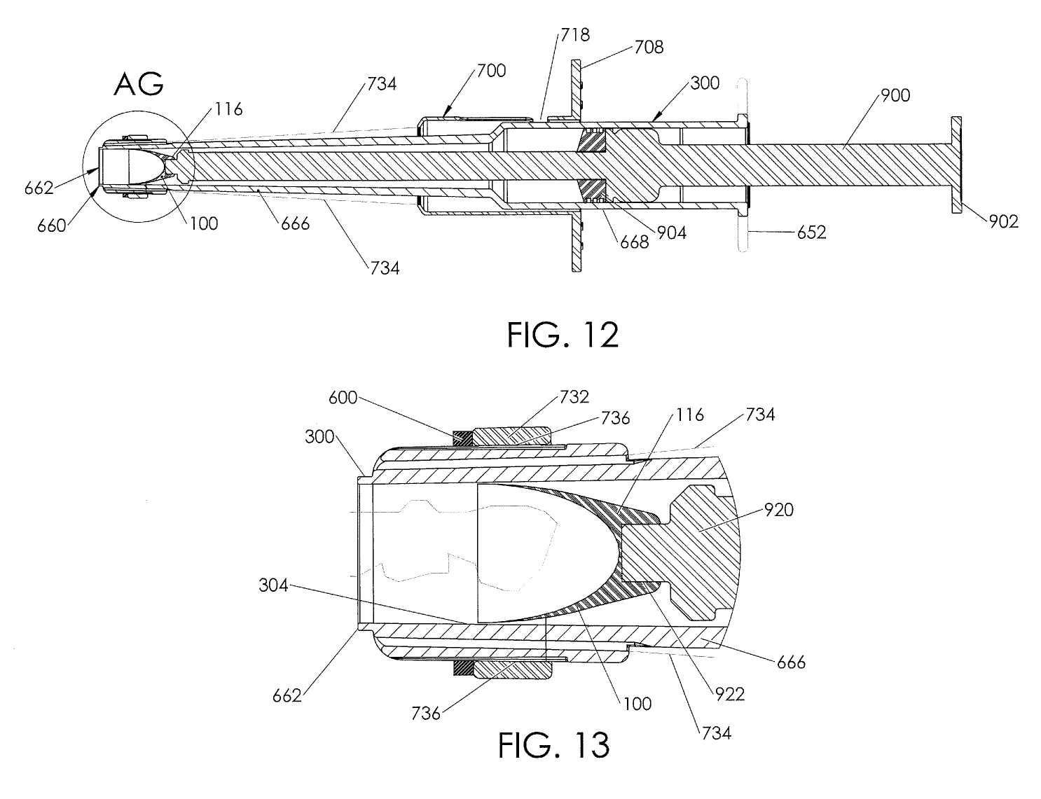

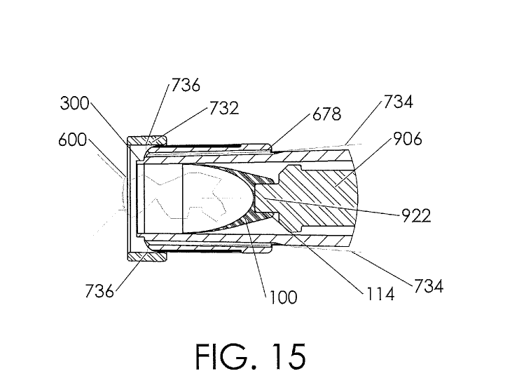

After insertion into a patient of ligation device 5b, plunger 900 may be withdrawn to pull hemorrhoid tissue into proximal opening 662. In operation ligation device 5b will be inserted into a patient in the locked position, which is depicted, for example in FIGS. 8, 9 and 16. Plunger 900 will be withdrawn while outer tubular member 700 is in the locked position shown in FIG. 15. This position is shown for example in FIGS. 12 and 13. Once plunger 900 has been withdrawn to the desired position, outer tubular member 700 is rotated so that it may be pulled distally relative to inner tubular member 650. Anchor 720 will move in leg 724 to allow the movement of outer tubular member 700. Tethers 734 will be pulled, which will urge pusher ring 732 proximally to urge band 600 from inner tubular member 650 to band tissue. This position is shown for example in FIGS. 15 and 17, in which the banding operation is complete. The current disclosure thus describes an embodiment capable of single hand use and ligation device.

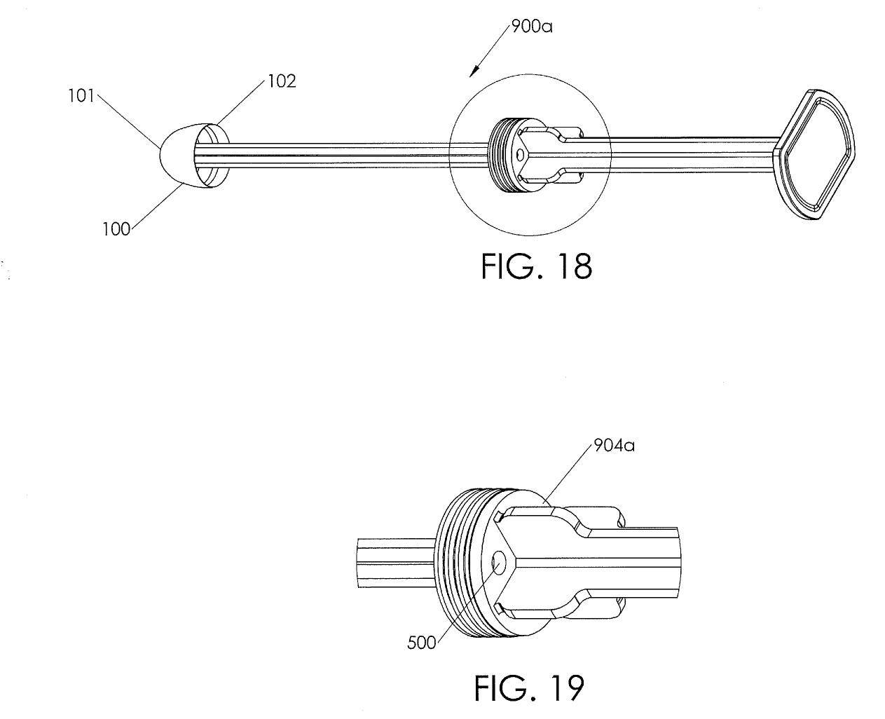

FIGS. 18 and 19 show an embodiment of a plunger 900a in which a valve opening 500 is disposed within a sealing means 904a.

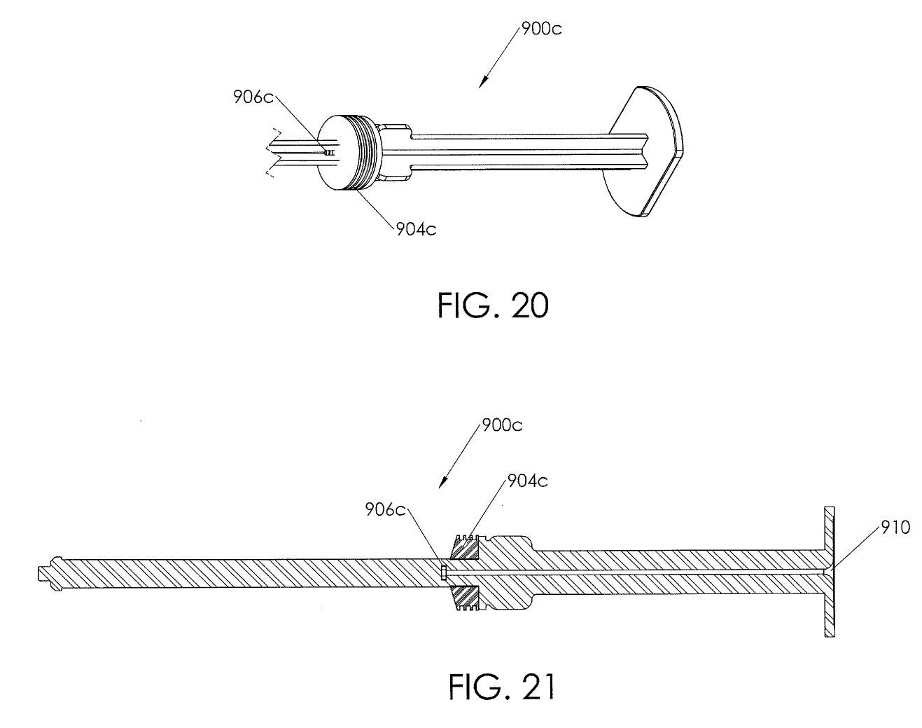

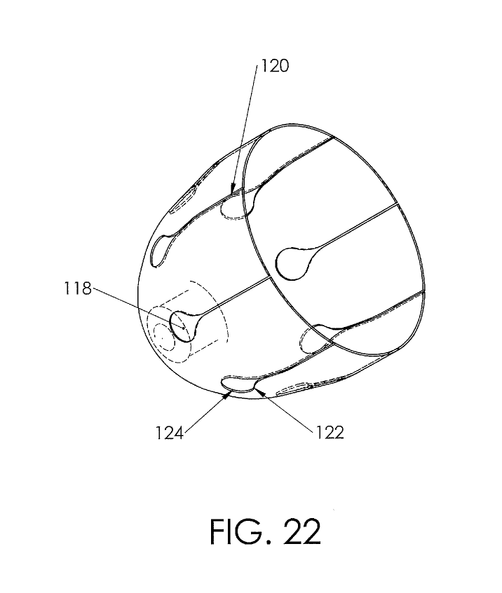

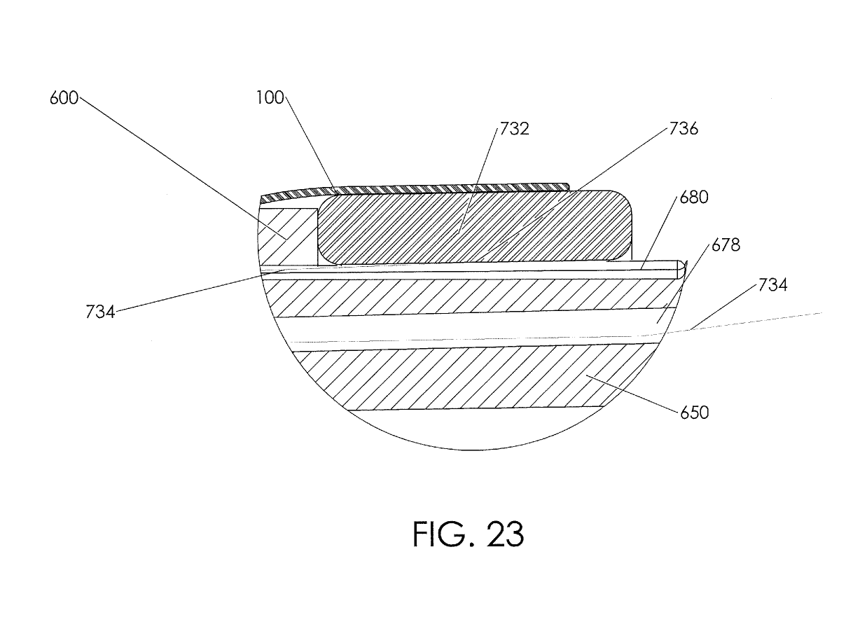

FIGS. 19 and 20 depict an alternative embodiment plunger 900c with a proximal stem vent hole 906c communicated with an opening 910 through a channel 912. A person skilled in the art would recognize that the location of the vent holes 906 is not critical to a successful embodiment. One object of this embodiment is to allow the operator to manually seal the opening 910c, thereby providing the operator with a means to develop a negative pressure above the negative pressure that is reached when the valve is open. The different embodiments of the plungers can be used with both embodiments of the ligation devices described herein.

A method of banding tissue may therefore comprise inserting a ligation device into the rectum of a patient and drawing tissue into the inner tubular member of the ligation device. The tissue is may be drawn into the inner tubular member by withdrawing an end of the plunger into the inner tubular member. The method may further comprise urging an elastic band off of the inner tubular member by moving the outer tubular member relative to the inner tubular member.

In one embodiment the method may comprise moving the outer tubular member distally to move the elastic band in the proximal direction and off of the proximal end of the inner tubular member. The step of moving the elastic band may comprise moving a pusher ring disposed on the inner tubular member into the elastic band. The method may be performed using a single hand. The outer tubular member may be connected with a tether, or other connector, so that the distal movement of the outer tubular member translates into proximal movement of the pusher sleeve. The method may further comprise moving the ligation device from a locked to an unlocked position prior to withdrawing the plunger. The unlocking step may comprise rotating the outer tubular member so that an anchor is movable longitudinally in a locking slot to allow distal movement of the outer tubular member relative to the inner tubular member. The steps of withdrawing the plunger and moving the outer tubular member may be performed with one hand while allowing the operator to perform other necessary tasks with a free hand.

Thus, it is seen that the apparatus and methods of the present invention readily achieve the ends and advantages mentioned as well as those inherent therein. While certain preferred embodiments of the invention have been illustrated and described for purposes of the present disclosure, numerous changes in the arrangement and construction of parts and steps may be made by those skilled in the art, which changes are encompassed within the scope and spirit of the present invention.

What is claimed is:

1. An elastic band ligation device comprising:
an inner tubular member with first and second ends, the first end defining a first opening;
an elastic band disposed about the inner tubular member;
a plunger movably received in the inner tubular member, the plunger having first and second ends; and
an invertible insertion cap at the first end of the plunger for insertion into the rectum of a patient, wherein the insertion cap will invert and be pulled into the inner tubular member when the plunger is withdrawn to draw tissue into the inner tubular member.

2. The ligation device of claim 1 further comprising an outer tubular member disposed about the inner tubular member and configured so that moving the outer tubular member relative to the inner tubular member will urge the elastic band past the first end of the inner tubular member to band hemorrhoidal tissue.

3. The ligation device of claim 1 wherein in an insertion position the insertion cap covers the elastic band.

4. The ligation device of claim 1 further comprising:
a seal mounted on the plunger, the seal being sealingly and slidably engaged with an inner surface of the inner tubular member; and
a valve opening defined through the inner tubular member and positioned to limit the suction applied to a patient when the plunger is withdrawn in the inner tubular member.

5. The ligation device of claim 4, wherein the valve opening is communicated with an exterior of the ligation device.

6. The ligation device of claim 2, wherein moving the outer tubular member distally relative to the inner tubular member will urge the elastic band past the first end of the inner tubular member.

7. The ligation device of claim 6 further comprising:
a movable pusher ring disposed about the inner tubular member; and
a tether connecting the outer tubular member to the movable pusher ring, wherein moving the outer tubular member distally relative to the inner tubular member urges the movable pusher ring forward to push the elastic band off of the inner tubular member.

8. An elastic band ligation device comprising:
an inner tubular member configured for insertion into the rectum of a patient, the inner tubular member having first and second ends, the first end defining an opening therethrough;
an elastic band disposed about the inner tubular member;
an outer tubular member disposed about and slidable relative to the inner tubular member;
a plunger disposed in the inner tubular member configured to generate suction when pulled therethrough to draw tissue into the inner tubular member; and
a valve opening positioned to limit the suction generated by movement of the plunger.

9. The ligation device of claim 8, the inner tubular member comprising first and second sections wherein the first section is smaller in diameter than the second section, and wherein the plunger extends from the first to the second section, the ligation device further comprising a seal on the plunger configured to sealingly engage the inner surface of the inner tubular member and create suction through the first end of the inner tubular member when the plunger is pulled therethrough.

10. The ligation device of claim 9, the valve opening being defined in the inner tubular member and positioned forward of the seal and communicated with an exterior of the ligation device.

11. The ligation device of claim 9, the valve opening being defined in a seal body of the seal and communicated with an exterior of the ligation device.

12. The ligation device of claim 8, further comprising a movable pusher ring disposed about the inner tubular member adjacent the elastic band, wherein distal movement of the outer tubular member relative to the inner tubular member moves the pusher ring and elastic band proximally.

13. The elastic band ligation device of claim 8, further comprising an insertion cap at the first end of the plunger thereof, the insertion cap being invertible from an insertion to a retracted position in which the insertion cap is received in the inner tubular member.

14. An elastic band ligation device comprising:
an inner tubular member insertable into a rectum of a patient;
an elastic band disposed about the inner tubular member;
a plunger movably disposed in the inner tubular member;
a seal on the plunger disposed in the inner tubular member and slidably engaged with an inner surface thereof;
an insertion cap at a first end of the plunger, wherein the plunger is insertable in to the rectum of a patient; and
an outer tubular member disposed about the inner tubular member, wherein movement of the outer tubular member distally relative to the inner tubular member will urge the elastic band off of the proximal end of the inner tubular member.

15. The ligation device of claim 14 further comprising a pusher ring disposed about the inner tubular member, wherein distal movement of the outer tubular member moves the pusher ring proximally to urge the elastic band off of the inner tubular member to band tissue.

16. The ligation device of claim 15 further comprising:
a tether connected to the pusher ring at one end and to the outer tubular member at a second end thereof.

17. The ligation device of claim 14, wherein the insertion cap is retracted into the inner tubular member when the plunger is pulled distally relative to the inner tubular member to pull hemorroidal tissue into the inner tubular member.

18. The ligation device of claim 17, wherein the insertion cap moves from an insertion to an inverted position when pulled into the inner tubular member.

19. The ligation device of claim 14 wherein the outer tubular member is movable from a locked position to an unlocked position in which the outer tubular member is movable longitudinally relative to the inner tubular member.

20. The ligation device of claim 14 further comprising.
an anchor extending from the inner tubular member; and
a locking slot defined in the outer tubular member, the anchor being received in the locking slot, wherein rotation of the outer tubular member moves the outer tubular member form a locked to an unlocked position in which the outer tubular member is movable longitudinally relative to the inner tubular member.
